(19) United States
(12) Patent Application Publication
(10) Pub. No.: US 2018/0153380 A1
(43) Pub. Date: Jun. 7, 2018

(54) ENDOSCOPIC SLEEVE WITH WINGS

(71) Applicant: Endoaid Ltd., Caesarea (IL)

(72) Inventors: Dan Rottenberg, Haifa (US); Omer Shezifi, Haifa (IL); Dima Pinhasov, Kiryat Yam (IL); Evgeny Kapilov, Carmiel (IL)

(73) Assignee: Endoaid Ltd., Caesarea (IL)

(21) Appl. No.: 15/575,370

(22) PCT Filed: May 16, 2016
(86) PCT No.: PCT/IB2016/052817

§ 371 Date: Nov. 19, 2017

Related U.S. Application Data
(60) Provisional application No. 62/163,450, filed on May 19, 2015.
(60) Provisional application No. 62/191,436, filed on Jul. 12, 2015.

(51) Int. Cl. A61B 1/00; A61B 1/31; A61B 1/01

(52) U.S. Cl. CPC A61B 1/00135; A61B 1/01; A61B 1/31

(57) ABSTRACT

An endoscopic sleeve includes a tubular member from which extend spaced projecting elements. The projecting elements are bendable towards both proximal and distal directions of the tubular member. The force (insertion force) required to bend the projecting elements towards the proximal direction is less than a force (extraction force) required to bend the projecting elements towards the distal direction. An outer periphery of the projecting elements decreases as the extraction force increases.

### FIELD OF THE INVENTION

The present invention relates to a sleeve or cuff having external projections, e.g., full or partial rings or wings, for use with medical endoscopes, particularly but not exclusively, a colonoscope.

### BACKGROUND OF THE INVENTION

In endoscopic examinations/procedures, flexible instruments are used to view a body lumen, such as the gastrointestinal tract and many others. The instruments are provided with fiber optic or charge-couple device (CCD) cameras which enable images to be transmitted around bends and images to be produced to displays on a screen.

For example, colonoscopic and enteroscopic examinations are the most effective techniques to assess the state of health of the bowel. However, they are inconvenient, uncomfortable, expensive procedures that are associated with significant risks of potentially serious complications. A further disadvantage is that they are time consuming for patients and medical personnel alike.

Further additional significant difficulties associated with colonoscopy and scoping procedures in general are as follows:

First, the anatomy of the colon lining has many folds. As the tip of the endoscope passes along the lumen of the colon, these folds hamper the endoscopist's ability to visualize the entire surface of the mucosa and in particular, to detect pre-malignant and malignant lesions tucked away on the proximal face of these folds during extubation.

Second, the position of the tip may be difficult to maintain from the moment at which a lesion or polyp is detected to the completion of any therapeutic procedure. As the colonoscope is moving the tip does not travel back at a constant speed but rather there are jerks and slippages, particularly when traversing a bend or length of colon where the bowel has been concertinaed over the endoscope shaft during intubation. The tip of the device may, at any moment, slip backwards thereby causing the clinician to lose the tip position. If tip position is lost, the clinician is required to relocate the lesion or polyp for the therapeutic procedure to be continued.

Third, bowel tissue is flexible and may fall over the scope distal end, disturbing the camera view/video picture.

Fourth, fecal and liquid remains may hide the colon walls, preventing proper examination of the colon tissue.

The colonoscopic procedure is not simple because the bowel is long and convoluted. In places it is tethered by peritoneal bands and in others it lies relatively free. When the tip of the endoscope encounters a tight bend the free part of the colon “loops” as more of the endoscope is introduced and causes difficulty to negotiate the bend.

PCT Patent Application WO 2011/148172 and Japan Patent Application 20022149540 both describe a sleeve for a medical scope distal section. The sleeve has a plurality of moveable, external, angled projecting elements having a tip and a base, which are moveable between a resting angled position to a position wherein the tip of the projecting element is substantially parallel to a longitudinal axis of the medical scope, and to a position that is at an angle approximately perpendicular to the longitudinal axis of the medical scope. The device is intended to close the projecting elements while the medical scope is moving forward (distally), and open the projecting elements during withdrawal of the medical scope (proximally), thereby to assist opening colon folds for better colon mucosa examination during scope withdrawal (only). However, there is a problem with the above solution. Since bowel screening is not usually done in one withdrawal movement but in short movements backwards and forward, such projecting elements may not reach a perpendicular position relative to the longitudinal axis of the medical scope.

PCT Patent Application WO 00/13736 describes an apparatus for percutaneous insertion into the cardiovascular system. It includes a catheter or catheter guide having a distal end, and flexible, permanently extended, generally radial protrusions (e.g., thin flexible fins or radially spaced fins) situated adjacent the distal tip of the catheter.

PCT Patent Application PCT/US2013/044407 describes an endoscopic sleeve that includes a tubular member from which extend a plurality of spaced projecting elements. The projecting elements are bendable towards both proximal and distal directions of the tubular member. The force (insertion force) required to bend the projecting elements towards the proximal direction is less than a force (extraction force) required to bend the projecting elements towards the distal direction. The projecting elements may be multiple thin rings (partial or full) or wings that are arranged circumferentially around the sleeve and along the length of the sleeve, abutting or leaning on each other during scope withdrawal to increase the bending force during colon screening.

### SUMMARY

Reference herein to a “medical scoping device” is intended to encompass endoscopes, enteroscopes, gastroscopes, colonoscopes and other types of scopes, and is used interchangeably and is intended to include all scoping instruments inserted into or through a body/organ/tissue lumen or cavity (used interchangeably). Endoscopy involves the inspection and treatment of the inside of the body lumen or cavity.

There is provided in accordance with an embodiment of the invention, several endoscopic sleeves, each of which includes a tubular member from which extend a plurality of spaced projecting elements. The projecting elements are bendable or movable towards both proximal and distal directions of the tubular member. The force (insertion force) required to bend the projecting elements towards the proximal direction is less than a force (extraction force) required to bend the projecting elements towards the distal direction. The projecting elements may be more bendable towards the proximal direction than towards the distal direction.

Some of the sleeve projecting elements may be moveable between at least three positions. In a first position, the projecting elements protrude freely, at an angle, such as perpendicular, to the longitudinal axis of the endoscope (so called “resting position”). In a second position, when the sleeved endoscope is introduced distally into a body lumen, insertion forces act upon the thin projecting elements to push them proximally backwards towards the shaft of the endoscope so that they may become tilted or even substantially parallel to the longitudinal axis of the endoscope, reducing the total device and sleeve diameters. In a third position, when the endoscope is withdrawn in a proximal direction out of the patient lumen, the thin projecting elements are bent by extraction forces, this time to the other direction (distally). The projecting elements fan out and extend from the shaft of the endoscope so as to gently contact or grip the inner surface of the body lumen. During extraction, the total device and sleeve diameters also may increase.

The projecting elements may be multiple thin rings (partial or full) or wings that are arranged circumferentially around the sleeve and along the length of the sleeve. Without limitation, there may be between 1 and 30 projecting elements. It will be appreciated that the projecting elements may, in some embodiments, be provided as a single ring. Each projecting element may have the same thickness, or different projecting elements may have different thicknesses. The projecting element may have a variable thickness along its extended outward diameter or its perimeter. Without limitation, each projecting element can have an outer diameter between 20 to 60 mm, and more preferably between 30 to 50 mm, with a thickness between 0.2 to 2.0 mm, and more preferably between 0.3 to 1.0 mm. All projecting elements may have the same diameter, or different projecting elements may have different diameters, or any single projecting element can have a changeable diameter. Projecting elements may be spaced apart by a distance of between 1 to 10 mm and more preferably 2 mm to 5 mm. Different sizes of gaps may be used for different projecting elements.

### BRIEF DESCRIPTION OF THE DRAWINGS

Embodiments of the invention are further described hereinafter with reference to the accompanying drawings, in which:

FIG. 1 is a simplified illustration of an endoscopic sleeve, constructed and operative in accordance with an embodiment of the invention, mounted on an endoscope and inserted in a body lumen;

FIGS. 2A and 2B are simplified illustrations of the endoscopic sleeve and endoscope, respectively during distal motion and proximal motion of the endoscope in the body lumen, in accordance with an embodiment of the invention;

FIGS. 3A and 3B are simplified illustrations of an endoscopic sleeve, constructed and operative in accordance with an embodiment of the invention, wherein projecting elements of the endoscopic sleeve are limited in forward (distal) bending by bulges positioned distally to the rings or wings.

FIG. 4 is a simplified illustration of an endoscopic sleeve, constructed and operative in accordance with another embodiment of the invention, including projecting elements (wings) having variable diameter.

FIG. 5 is a simplified illustration of an endoscopic sleeve having one projecting element constructed as continuous ring having a screw shape.

FIG. 6 simplified illustration of an endoscopic sleeve having one projecting element build as continuous variable diameter ring having a screw shape.

FIG. 7 is a simplified illustration of an endoscopic sleeve, constructed and operative in accordance with another embodiment of the invention, in which projecting elements are rolled wings.

FIG. 8 is a simplified illustration of an endoscopic sleeve, constructed and operative in accordance with another embodiment of the invention, in which flexible arches are connected to the sleeve tubular member on both sides.

FIG. 9 is a simplified illustration of an endoscopic sleeve, constructed and operative in accordance with another embodiment of the invention, in which the projecting elements are movable axially over the endoscope.

FIG. 10 is a simplified illustration of an endoscopic sleeve, constructed and operative in accordance with another embodiment of the invention, in which the wings are folded by an axially sliding cover.

FIG. 11 is a simplified illustration of an endoscopic sleeve, constructed and operative in accordance with another embodiment of the invention, in which the wings are tied with cords that limit their distal bending.

FIG. 12 is a simplified illustration of an endoscopic sleeve cover used to cover the endoscopic sleeve of the invention, and which can be removed by pulling a cord.

FIG. 13 is a simplified illustration of an endoscopic sleeve, constructed and operative in accordance with another embodiment of the invention, in which a peel-away strip is incorporated in the main tubular member sleeve for easy removal of the sleeve.

FIG. 14 is a simplified illustration of an endoscopic sleeve, constructed and operative in accordance with another embodiment of the invention, in which small inner angled fins are part of the main tubular member sleeve, wherein the fins reduce the sleeve assembly force and increase the sleeve removal force.

FIG. 15 is a simplified illustration of an endoscopic sleeve, assembled over an endoscope, constructed and operative in accordance with an embodiment of the invention.

FIGS. 16A and 16B are simplified illustrations of the endoscopic sleeve tubular member having several deep grooves, mounted on small diameter and large diameter endoscopes.

FIG. 17 is a simplified illustration of an endoscopic sleeve, constructed and operative in accordance with an embodiment of the invention, wherein a sealing ring is mounted over the distal section of the sleeve.

FIGS. 18A and 18B are simplified illustrations of an endoscopic sleeve, having a slotted tubular member distal section, mounted freely over an endoscope, and locked in place using a sealing ring.

FIGS. 19A and 19B are simplified illustrations of an endoscopic sleeve, in which the first wing layer is separate from the sleeve and can be used as a tightening ring.

### DETAILED DESCRIPTION

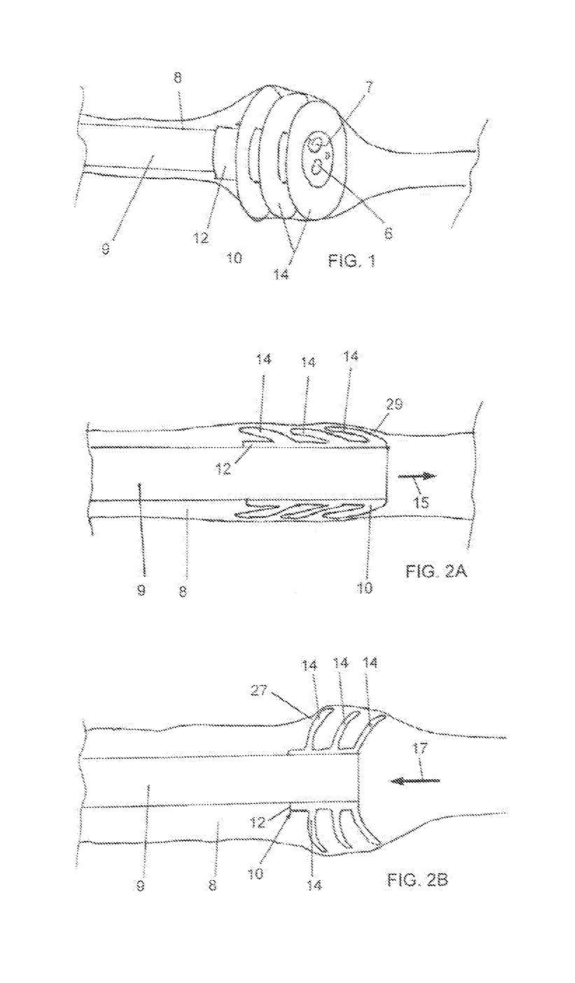

Reference is now made to FIG. 1, which illustrates an endoscopic sleeve 10, constructed and operative in accordance with an embodiment of the invention, mounted on an endoscope 9 and inserted in a body lumen 8, such as but not limited to, the colon or other parts of the GI tract or other body lumens. Endoscope 9 has one or more image capturing devices 7 for viewing the body lumen and working lumens 6 (such as for introducing tools to collect tissue samples, or for irrigation or suction, etc.), as is well known in the art.

Sleeve 10 is arranged for mounting over the distal end of the shaft of endoscope 9 so as to surround (or partially surround) and extend along at least a distal part or tip region of the endoscope shaft.

In a non-limiting embodiment of the invention, endoscopic sleeve 10 includes a tubular member 12 from which extend a plurality of spaced projecting elements 14. Projecting elements 14 are bendable towards both proximal and distal directions of tubular member 12. As will be explained further herein below, projecting elements 14 are more bendable towards the proximal direction than towards the distal direction. In the illustrated embodiment, projecting elements 14 are full rings, but can be partial rings or wings, and may be initially generally perpendicular to tubular member 12.

All components of sleeve 10 are constructed of a suitable biocompatible material so that they are flexible, resilient and deformable. Examples of suitable materials include, but are not limited to, polymers, elastomers and rubbers, such as polyurethane, natural rubber, silicone and silicone elastomeric materials. The material is preferably transparent to be able to hold tissue and still allow visualization.

Tubular member 12 and projecting elements 14 may be made of the same material or different materials. Some of the projecting elements 14 may be made of different materials than other projecting elements 14.

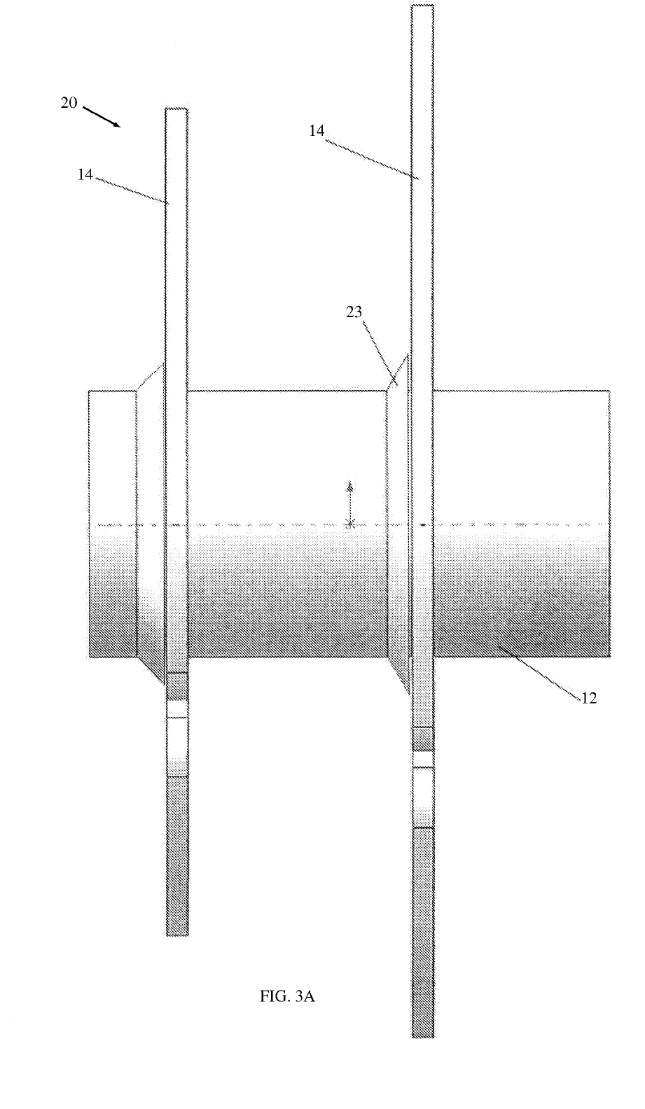

Reference is now made to FIG. 2A, which illustrates endoscopic sleeve 10 and endoscope 9 during distal motion (such as insertion) in the body lumen 8, as indicated by arrow 15. The projecting elements 14 bend backwards proximally and can be generally parallel to tubular member 12. In this manner, projecting elements 14 do not hinder distal progression of the endoscope in the body lumen.

In FIG. 2B, endoscopic sleeve 10 and endoscope 9 are moved proximally (such as during retraction or during reciprocating motion of the endoscope) in the body lumen 8, as indicated by arrow 17. During proximal motion of the endoscope in the body lumen, projecting elements 14 sufficiently project away from tubular member 12 so as to contact and unfold tissue folds in body lumen 8 for improved endoscopic visualization and screening of the folds.

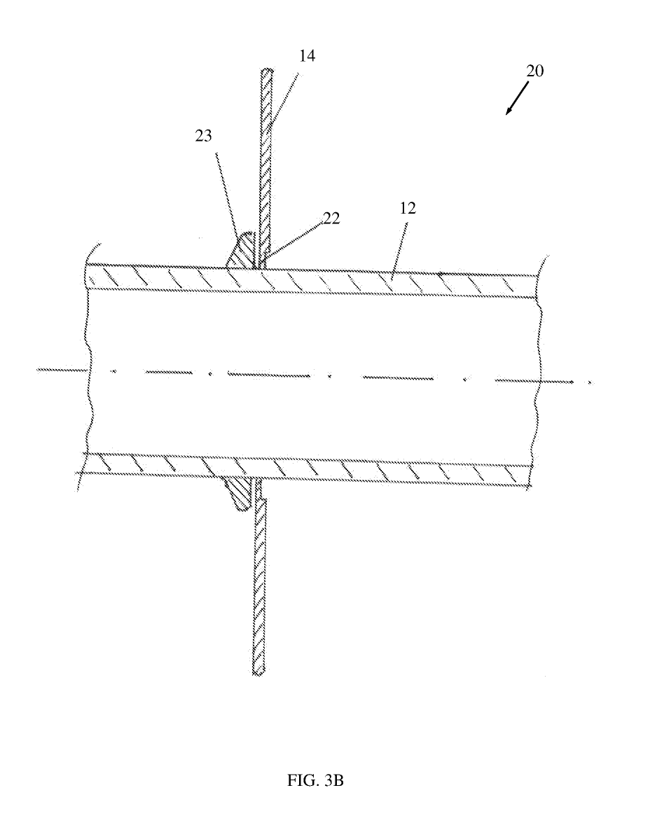

Reference is now made to FIGS. 3A-3B, which illustrate an endoscopic sleeve 20, constructed and operative in accordance with an embodiment of the invention. In the illustrated embodiment in FIG. 3A, bulges 23 may be disposed around tubular member 12 distal to protrusion elements or wings 14. During proximal motion of the endoscope in the body lumen, protrusion elements or wings 14 abut against bulges 23, thereby increasing the bending resistance of protrusion elements 14 while screening the colon. The bulges 23 do not interfere with movement of the wings 14 during insertion into the colon, so that the bending forces are low during insertion of sleeve 20 into the colon.

FIG. 3B illustrates that all or some of the projecting elements or wings 14 may include a thin root portion 22 (i.e., thinner than the rest of the wing), whose height is smaller than bulges 23. This further reduces the required bending force during insertion of sleeve 20, but maintains the higher bending force during withdrawal of sleeve 20 and screening, which helps open the colon folds by the wing 14 leaning on bulge 23.

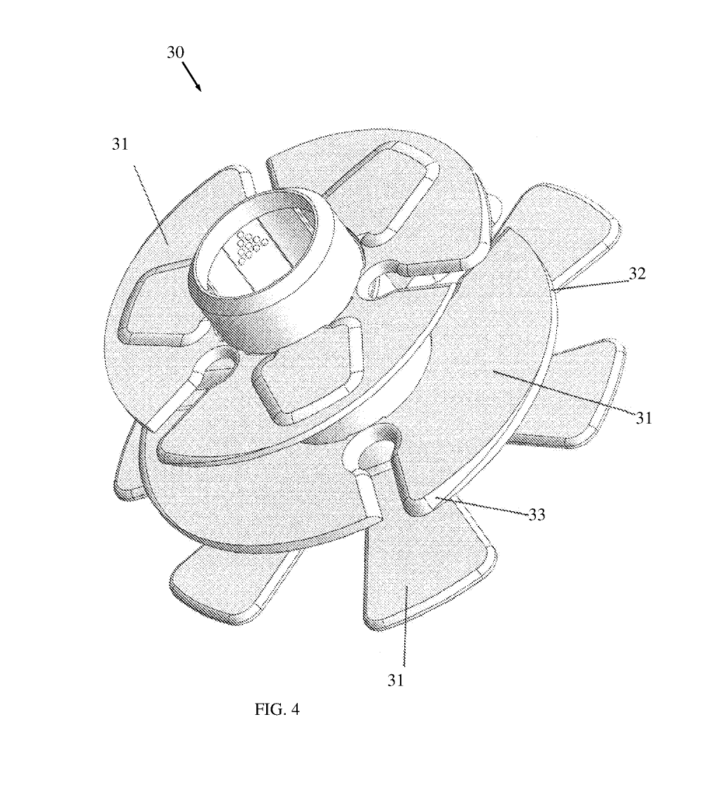

Reference is now made to FIG. 4, which illustrate an endoscopic sleeve 30, showing different types of projecting elements 31 having shapes of different wings and partial rings, which are more bendable towards the proximal direction than towards the distal direction. The projecting elements 31 may include combinations of any of the other embodiments described herein. For example, the structure of the embodiments of FIGS. 3A-3B may be used for rings, partial rings, wings and any other types of projecting elements, in the embodiment of FIG. 4.

The wings and partial rings 31 shown in FIG. 4 may have variable diameters, or different lengths, e.g., at one external end versus the other external end of the wing. For example, wing diameter at one end 32 of wing 31, may be smaller than the other end 33 of the wing 31. The advantages of this structure is to further reduce the bending resistance when the wings or partial rings 31 flip over when the endoscope moving direction is reversed, and to prevent the tips or ends of the wings 14 from clashing with one another during flipping so as to increase the required flipping force.

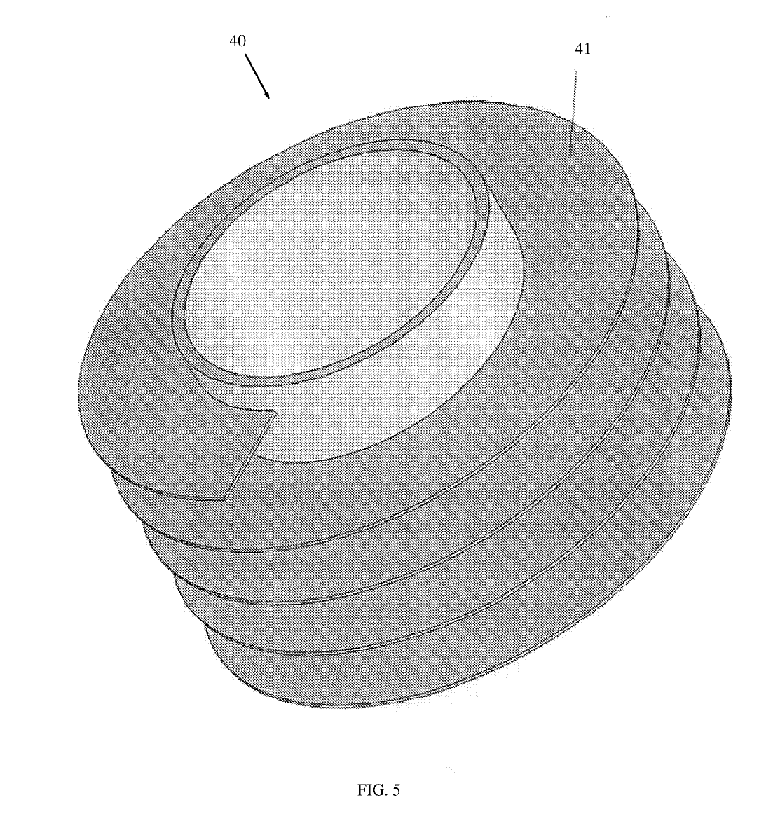

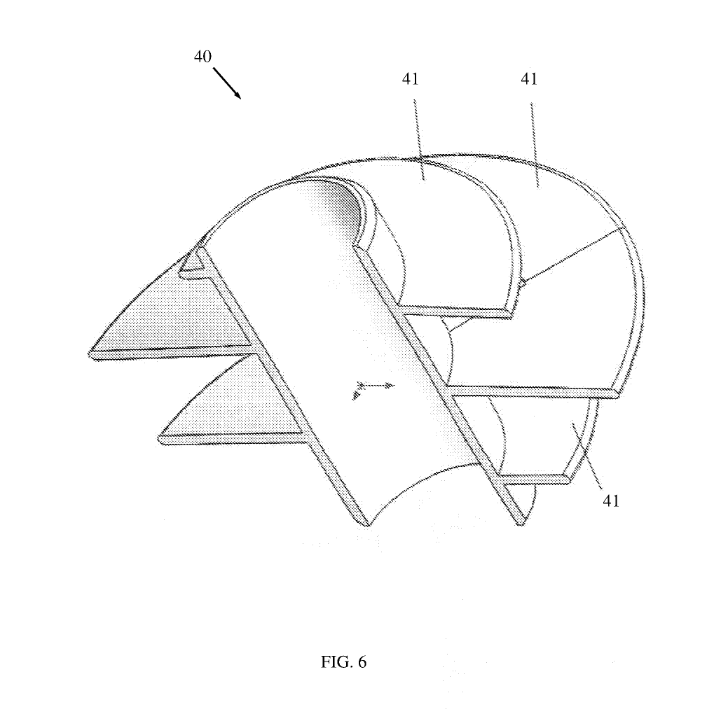

Reference is now made to FIG. 5, which illustrates an endoscopic sleeve 40, constructed and operative in accordance with another embodiment of the invention. In this embodiment, the projecting element 41 is preferably one continuous ring having a screw shape. This shape enables moving the folds axially while rotating the scope, or releasing the folds from the wings 41 by rotating the scope. The continuous ring may have different diameters along the screw shape, as shown in FIG. 6. More than one continuous ring may be incorporated in the sleeve design.

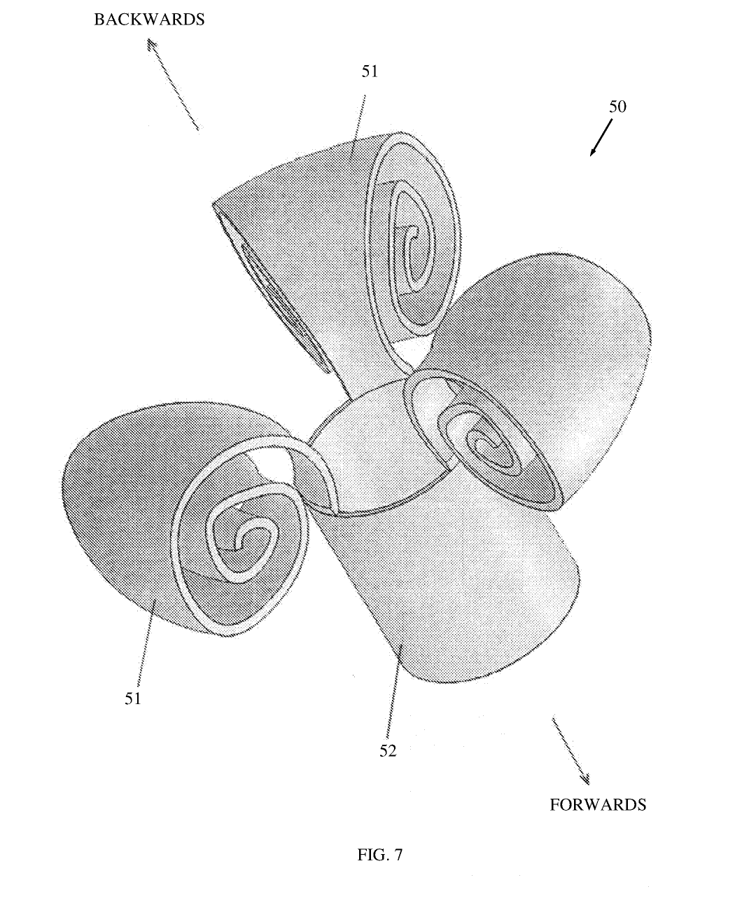

Reference is now made to FIG. 7, which illustrates an endoscopic sleeve 50, constructed and operative in accordance with another embodiment of the invention. In this embodiment, projecting elements 51 are rolled wings, which in their free position tend to roll like a spring. When the endoscope is moving backwards during colon screening, wings 51 roll up (that is, more and more layers of the wings overlap each other in the roll) so as to increase the sleeve 50 diameter and help open the colon folds. When the scope is moved forward, during scope insertion, friction with the colon walls opens the rolled wings, changing their shape to flat long wings positioned parallel to tubular member 52, thereby reducing the sleeve diameter and the resistance to scope insertion.

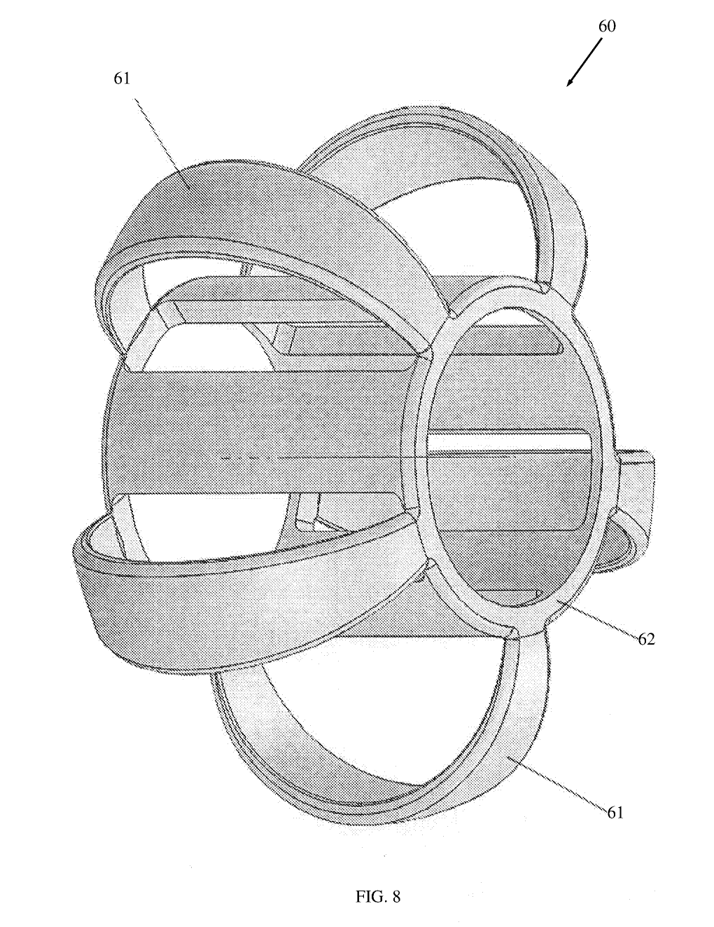

Reference is now made to FIG. 8, which illustrates an endoscopic sleeve 60, constructed and operative in accordance with another embodiment of the invention. In this embodiment, projecting elements 61 are flexible arches connected to tubular member 62 on both sides.

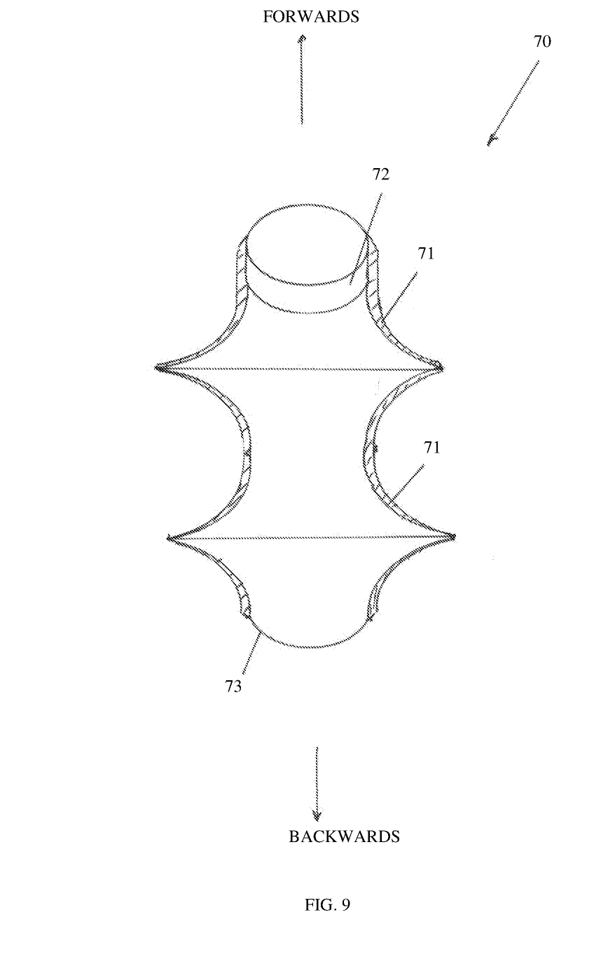

Reference is now made to FIG. 9, which illustrates an endoscopic flexible sleeve 70, constructed and operative in accordance with another embodiment of the invention. Sleeve 70 includes projecting elements 71 constructed like accordion layers or bellows, which can be stretched to reduce their diameter or squeezed to increase their diameter. The distal end of projecting elements 71 (which may be in the shape of a ring, as shown) is firmly attached to the endoscope distal tip, as opposed to the proximal end 73 which is free to move and can slide over the endoscope. During scope insertion (moving forward into the colon), projecting elements 71 are pushed back due to friction with the colon, and the diameter of sleeve 70 becomes smaller to reduce the insertion resistance into the scope. During backwards movement (screening), projecting elements 71 slide backwards and increase their diameter to open the colon folds.

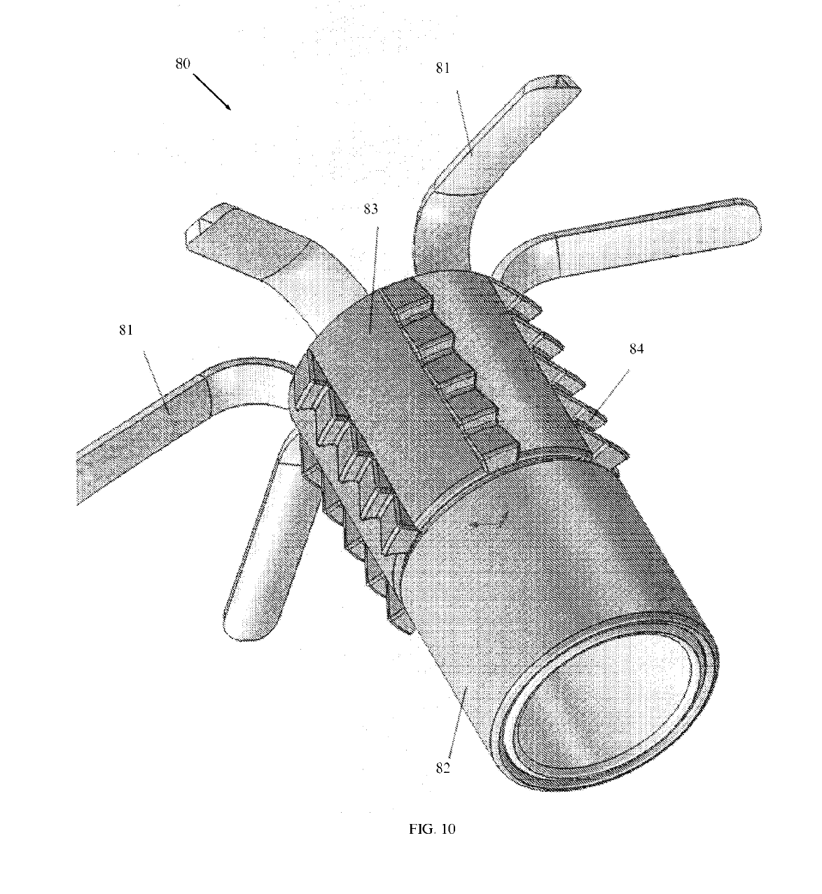

Reference is now made to FIG. 10, which illustrates an endoscopic sleeve 80, constructed and operative in accordance with another embodiment of the invention. Tubular member 82 is attached to the endoscope distal end. Sleeve 80 includes projecting elements 81 that are folded inside a tubular sliding member 83 during scope insertion. Sliding member 83 may be formed with peripheral friction teeth 84. The teeth 84 may catch and snag on the colon during scope insertion, so that sliding member 83 moves backward over projecting elements 81 which causes the projecting elements 81 to move inside tubular member 83, thereby decreasing the diameter of the device to make insertion easier. During scope withdrawal (screening), slider 83 slides distally, expose projecting elements 81 so they expand to a larger diameter, in order to “iron” (open and flatten) the colon folds and center the scope.

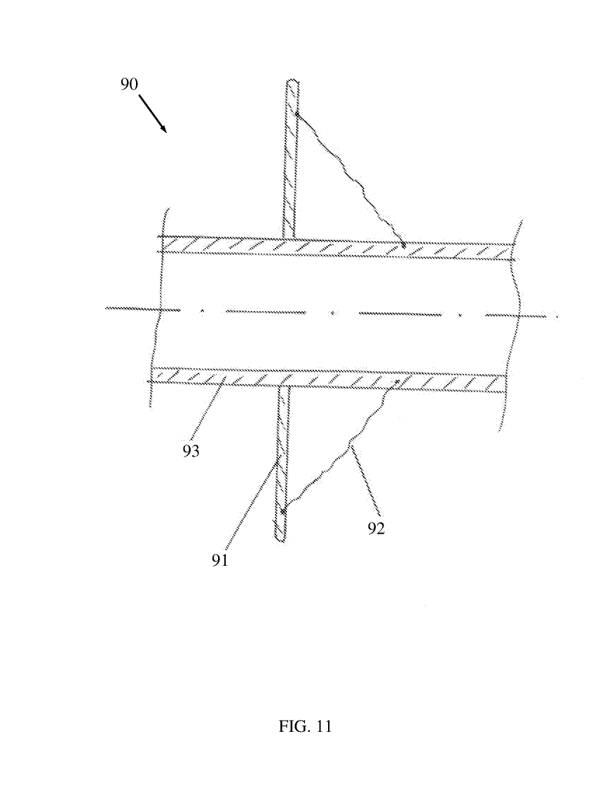

Reference is now made to FIG. 11, which illustrates an endoscopic sleeve 90, constructed and operative in accordance with another embodiment of the invention. Sleeve 90 includes projecting elements 91 that are very flexible and bend easily to both sides. To ensure enough folding resistance during screening to iron the folds, a cord 92 (or several cords) are tied between every projecting element or wing 91 and tubular member 93, to restrict the projecting elements banding motion during scope withdrawal. The term “cord” encompasses any thin element, such as but not limited to, a cord, band, wire, rod, string, filament, etc. For example, cord 92 can include a very thin, bendable layer of the same material of which sleeve 90 and projecting elements 91 are made.

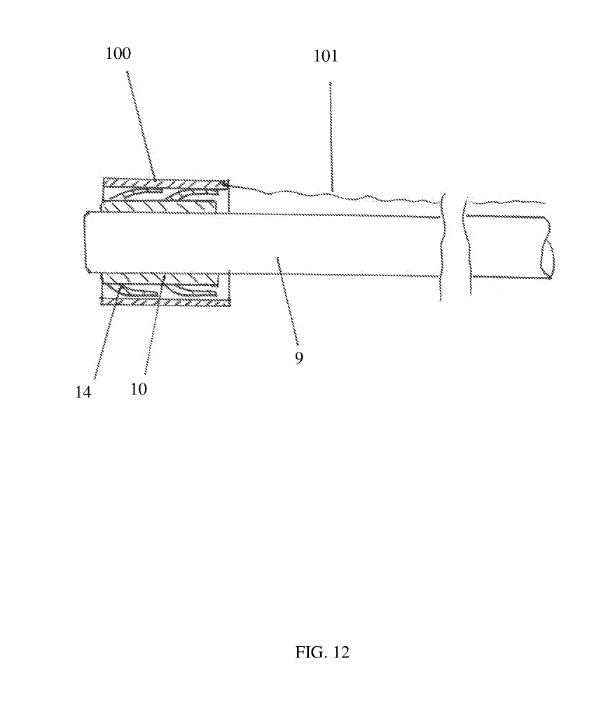

Reference is now made to FIG. 12, which illustrates an endoscopic sleeve cover 100, constructed and operative in accordance with another embodiment of the invention. The sleeve cover 100 can be used with any of the sleeves described above. Sleeve cover 100 is a tube, preferably made from a thin elastomer, which is manually mounted over the sleeve. Sleeve cover 100 covers the endoscope sleeve while the projecting elements are folded in the direction suitable for scope insertion into the colon. The sleeve cover 100 is stiff enough to fully fold the sleeve projecting elements, for example, projecting elements 14 of FIG. 1, to minimize the sleeve diameter during scope insertion. A long pulling cord 101 may be attached to sleeve cover 100. Cord 101 is inserted into the colon in parallel to the endoscope, and extended out of the patient colon. When scope insertion is completed, the physician may pull cord 101 to move sleeve cover 100 backwards and expose projecting elements 14, which then expand outwards to open and iron folds during scope withdrawal and colon screening.

In another embodiment of the invention, cover sleeve 100 can be a long sleeve assembled over the full working length of the endoscope. Even without a pulling cord, cover sleeve 100 can be pulled directly backwards manually to open the projecting elements before scope withdrawal.

In another embodiment of the invention, cover sleeve 100 can be attached to pulling cord at its distal end, and the pulling cord 101 can be threaded through the endoscope working channel 6 (see FIG. 1). Before scope withdrawal and screening starts, the pulling cord is pulled to pull sleeve cover distally into and through the scope working channel, so as to free the projecting elements to expand and iron the colon folds and center the scope inside the colon.

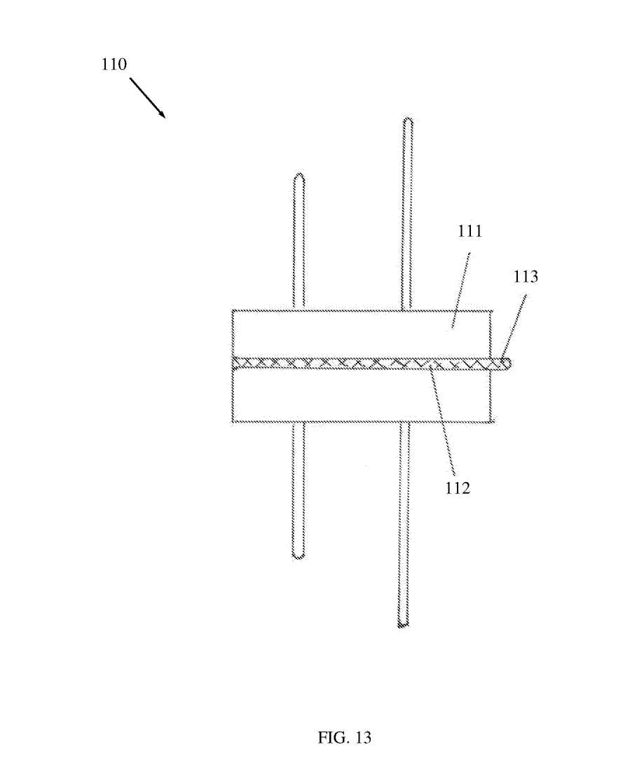

Reference is now made to FIG. 13, which illustrates an endoscopic sleeve 110, constructed and operative in accordance with another embodiment of the invention. Sleeve 110 has a tubular member 111 that includes a peel-away strip 112, added to allow easy removal of sleeve 110. The peel-away strip 112 may include a protruded section 113 that can be pulled manually at the end of the procedure in order to start the peel-away action, tear the peel away strip 112, and easily remove sleeve 110 from the endoscope.

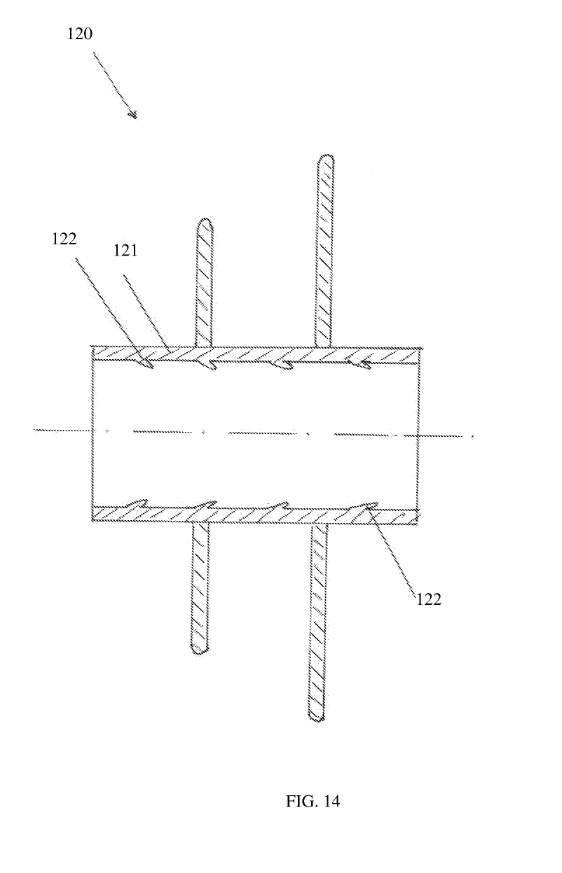

Reference is now made to FIG. 14, which illustrates an endoscopic sleeve 120, constructed and operative in accordance with another embodiment of the invention. Sleeve 120 includes a tubular member 121, which includes small inner angled fins 122. The fins 122 allow easy assembly of sleeve 120 over the endoscope distal tip, but increase the force required to remove sleeve 120. This is due to the fact that in order to remove sleeve 120 the inner fins 122 must flip over, which significantly increases the withdrawal force required to remove sleeve 120.

In another embodiment of the invention, the sleeve of the invention can be assembled using an assembly tool. The assembly tool may be inserted through the sleeve tubular member, and then expanded radially for easy assembly over the scope. After partial assembly of the proximal sleeve side, the sleeve may be held manually while the tool is pulled back, thereby decreasing the main tube diameter of the elastic sleeve to that it fits tight and snugly on the endoscope distal end.

In all embodiments of the invention, the sleeve, including its tubular member, may be made from flexible polymers, and the material flexibility helps in the attachment of the sleeve to the scope.

Since there are many types of scope sizes or diameters, the tubular member may be too tight on large scopes and too loose on small scopes. Different sleeves having different tubular members with different inner diameters may be provided to cover the large endoscope diameters range. This may be confusing for the user, who may choose the wrong type of sleeve, thereby causing the sleeve to fall off the scope during the procedure. Another disadvantage is that it may be very hard to assemble the wrong sleeve on the scope, potentially damaging the scope tip.

Accordingly, several solutions are presented herein to allow using one type or fewer types of sleeves and tubular members for all or most endoscopes diameters. The solutions include a tubular member with deep elastic grooves, a tubular member with different (more flexible or stiffer) ring attachments, a slotted/grooved tubular member and a slotted tubular member with an O-ring or a flexible, flat ring attachment.

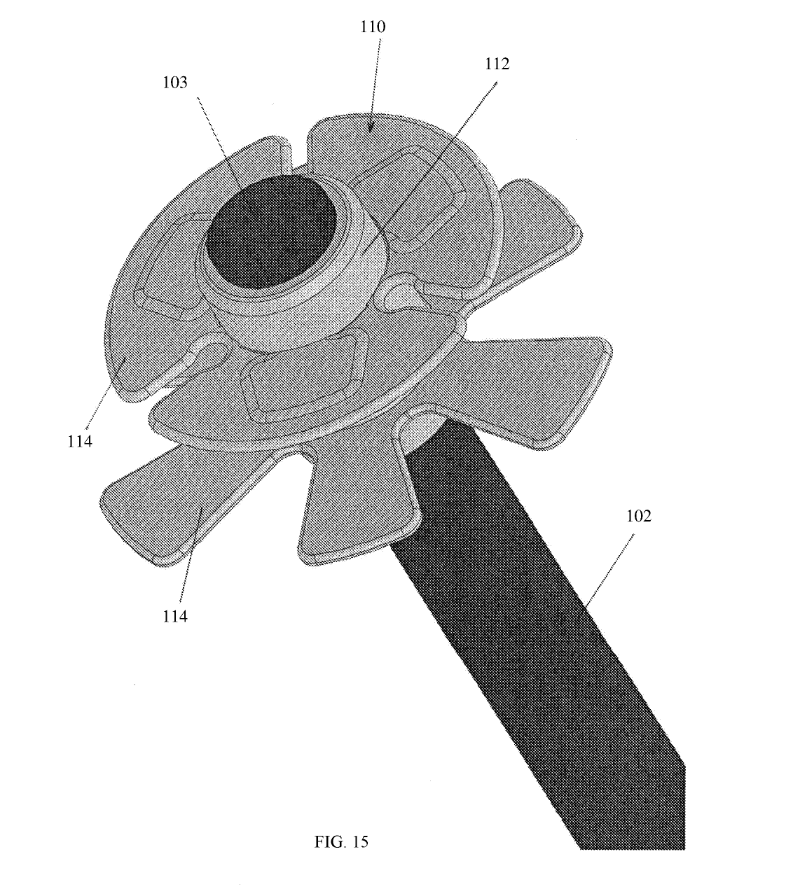

Reference is now made to FIG. 15, which illustrates an endoscopic sleeve 110, constructed and operative in accordance with an embodiment of the invention, mounted on an endoscope 102.

Sleeve 110 is arranged for mounting over the distal end of the shaft of endoscope 102 so as to surround and extend along at least a distal part or tip region 103 of the endoscope shaft.

In a non-limiting embodiment of the invention, endoscopic sleeve 110 includes a tubular member 112 from which extend a plurality of spaced projecting elements/wings 114. Projecting elements 114 are bendable towards both proximal and distal directions of tubular member 112.

All components of sleeve 110 are constructed of a suitable biocompatible material so that they are flexible, resilient and deformable. Examples of suitable materials include, but are not limited to, polymers, elastomers and rubbers, such as polyurethane, natural rubber, silicone and silicone elastomeric materials. The material is preferably transparent to be able to hold tissue and still allow visualization.

Tubular member 112 and projecting elements or wings 114 may be made of the same material or different materials. Some of the projecting elements 114 may be made of different materials than other projecting elements 114.

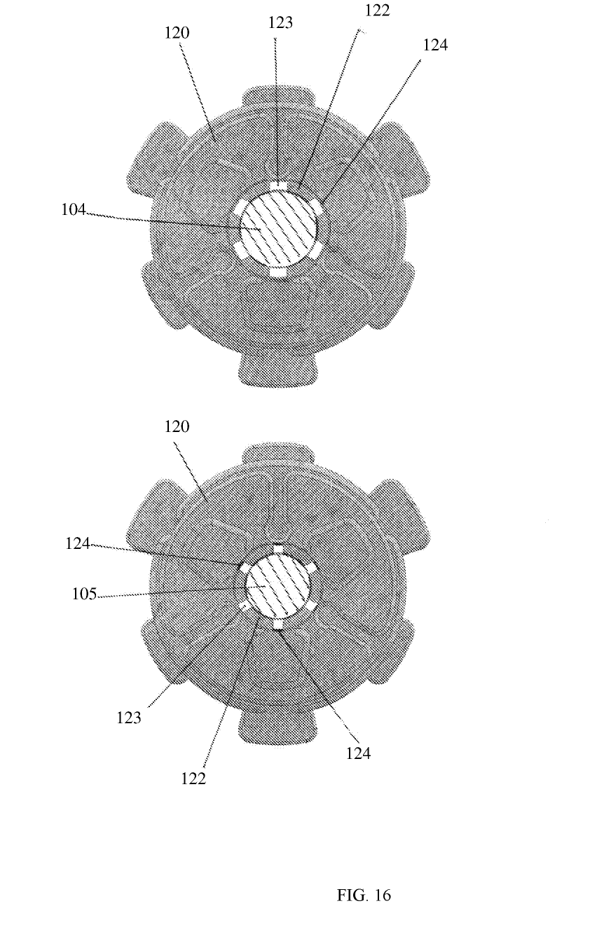

Reference is now made to FIG. 16A, which illustrates a sleeve 120 including a tubular member 122 mounted on a relative small diameter endoscope 105. The tubular member 122 of sleeve 120 may be formed with one or more grooves 123 (3 to 6 grooves may be sufficient).

Grooves 123 are relatively deep, preferably leaving a thin layer 124 of elastic material of tubular member 122 at the grooves location, having a thickness of less than 0.5 mm, and more preferably having thickness of 0.2-0.3 mm, so the thin layers 124 can easily stretch when a radial force is applied on tubular member 122 during assembly over an endoscope.

When sleeve 120 is mounted on a large diameter endoscope 104 (FIG. 16B), the elastic material in the thin remaining layer 124 at the base of grooves 123 stretches to accommodate assembly over the larger endoscope 105.

Although it may be possible to stretch the whole circumference of the tubular member 122 even without grooves 123, nevertheless the force required to stretch the whole circumference of the tubular member 122 over a large diameter endoscope is very large, making such assembly very difficult and may possibly cause damage to the endoscope sensitive distal tip. In contrast, the grooves 123 make the assembly much easier with no danger of damaging the sensitive distal tip.

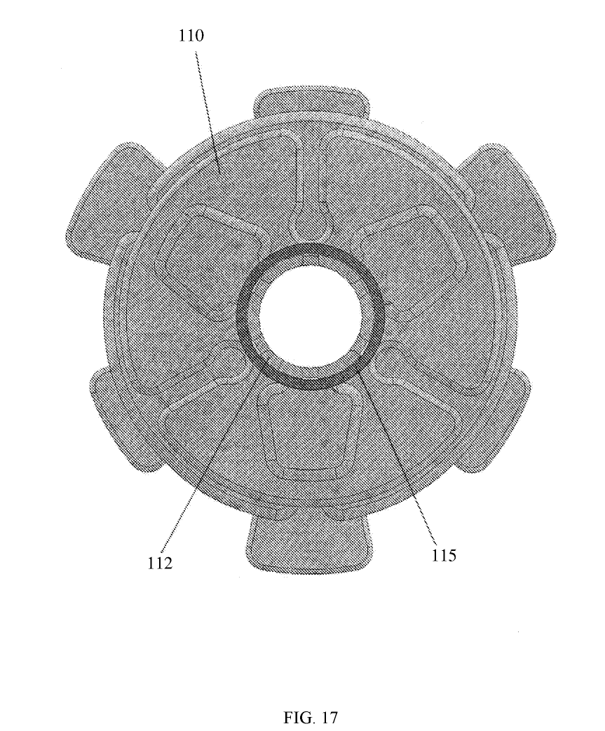

Reference is now made to FIG. 17, which illustrates endoscopic sleeve 110, wherein the inner diameter of tubular member 112 is relative large, so that tubular member 112 moves freely even over a large diameter endoscope. A smaller diameter sealing ring (e.g., O-ring or flat ring) 115 may be assembled over the distal section of tubular member 112, after sleeve 110 has been freely assembled on the endoscope distal section. The sealing ring 115 becomes squeezed towards the endoscope, thereby increasing the friction force between tubular member 112 and endoscope 103. The elastomeric material of sealing ring 115 is much stronger and stiffer than the sleeve material, and has a smaller inner diameter than the outer diameter of tubular member 112, thereby applying enough force to firmly attach sleeve 110 over a wide range of endoscope diameters, and locking the sleeve position over the endoscope.

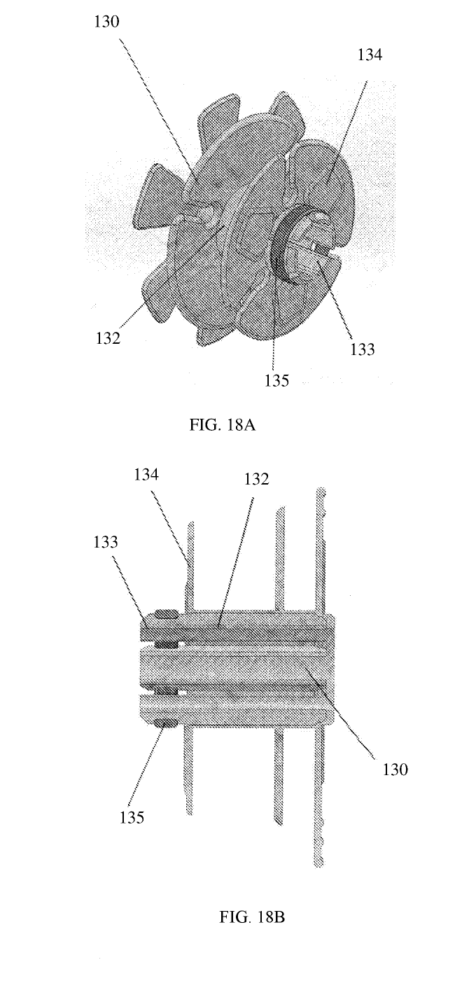

Reference is now made to FIGS. 18A and 18B, which illustrate an endoscopic sleeve 130 having a tubular member 132. The distal section of tubular member 132 may be cut or otherwise formed to create bulges 133, preferably, but not necessarily, equally disposed around the distal portion of tubular member 132. Bulges 133 may be positioned only in front of (distal to) protrusion elements or wings 134. The inner diameter of tubular member 132 is relative large so that tubular member 132 moves freely even over a large diameter endoscope. A smaller diameter sealing ring (e.g., flat-ring or O-ring) 135 may be assembled over bulges 133 at a distal portion of tubular member 132, after sleeve 130 has been freely assembled on the endoscope distal section. The sealing ring 135 squeezes bulges 133 towards the endoscope, and bends bulges 133 to match the endoscope distal section circumference. Sealing ring 135 is smaller in diameter than tubular member 132 and bulges 133. The elastomeric material of sealing ring 135 is stiffer and/or stronger than the sleeve material, so as to apply enough force to firmly attach sleeve 130 and bulges 133 over a wide range of endoscopes diameters.

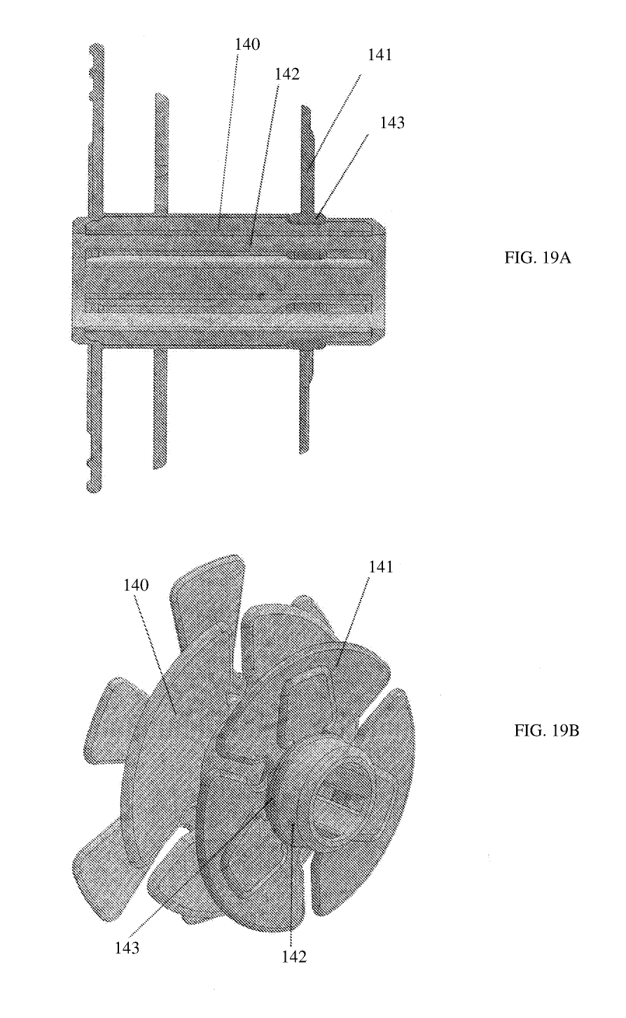

Reference is now made to FIGS. 19A and 19B, which illustrate an endoscopic sleeve 140, constructed and operative in accordance with another embodiment of the invention. Sleeve 40 may include a front layer of wings 141, which is separate from sleeve 140 and which is made from a stiffer and stronger elastomer than that of sleeve 140.

Sleeve 140 has a tubular member 142, whose inner diameter is relative large so as to move freely even over a large diameter endoscope. The front layer of wings 141 may have a flat round base 143, which is assembled over the larger diameter distal section of tubular member 142, after sleeve 140 has been freely assembled on the endoscope distal section.

The elastomeric material of front wings layer 141 is stronger than the sleeve material and has a smaller inner diameter than the outer diameter of tubular member 142, so that the flat base 143 applies enough force to firmly attach sleeve 140 over a wide range of endoscope diameters.

1. A device comprising:
an endoscopic sleeve comprising a tubular member from which extend a plurality of spaced projecting elements, said projecting elements being bendable towards both proximal and distal directions of said tubular member, wherein a force (insertion force) required to bend said projecting elements towards the proximal direction is less than a force (extraction force) required to bend said projecting elements towards the distal direction and an outer periphery of said projecting elements decreases as the extraction force increases, and further comprising structure to increase resistance of said projecting elements to bend towards the distal direction.

2. The device according to claim 1, wherein said structure comprises bulges against which said projecting elements abut when bending towards the distal direction.

3. The device according to claim 1, wherein said structure comprises said projecting elements or wings having a variable diameter.

4. The device according to claim 1, wherein said structure comprises said projecting elements comprising at least one continuous screw shape, having a fixed or variable diameter.

5. The device according to claim 1, wherein said structure comprises said projecting elements comprising discrete rolling wings that flatten out during scope insertion and roll back during scope withdrawal.

6. The device according to claim 1, wherein said structure comprises said projecting elements comprising flexible arches connected to the sleeve tubular member.

7. The device according to claim 1, wherein said projecting elements are arranged to axially slide over the endoscope to increase or decrease the sleeve diameter.

8. The device according to claim 1, wherein said structure comprises an axially sliding cover and said projecting elements comprise bendable wings that are selectively covered or exposed by said axially sliding cover.

9. The device according to claim 1, wherein said structure comprises cords and said projecting elements are tied in one side with said cords that limit their bending distally.

10. The device according to claim 1, wherein said tubular member comprises a peel-away strip to allow quick removal from the endoscope.

11. The device according to claim 1, wherein the tubular member of the sleeve comprises inwardly angled fins.

12. The device according to claim 1, wherein the tubular member of the sleeve is formed with radially stretchable grooves.

13. The device according to claim 1, further comprising a tightening ring mountable in or on a portion of the tubular member of the sleeve.

14. The device according to claim 13, wherein said tightening ring is mounted over bulges formed in said tubular member.

15. The device according to claim 1, wherein said structure comprises a separate distal layer of additional projecting elements that project from a ring base, said additional projecting elements being stiffer than said first-mentioned projecting elements and having an inner diameter smaller than an outer diameter of said tubular member.
